(19) United States
(12) Patent Application Publication
(10) Pub. No.: US 2022/0062146 A1
(43) Pub. Date: Mar. 3, 2022

(54) COMPOSITIONS AND METHODS FOR REMOVING DENTAL CALCULI

(71) Applicant: Dennis C. Mynarcik, Huntington, NY (US)

(72) Inventor: Dennis C. Mynarcik, Huntington, NY (US)

(21) Appl. No.: 17/415,359

(22) PCT Filed: Dec. 17, 2019
(86) PCT No.: PCT/US19/66969

§ 371 Date: Jun. 17, 2021

Related U.S. Application Data
(60) Provisional application No. 62/780,565, filed on Dec. 17, 2018.

(51) Int. Cl. A61K 8/66; C12N 9/22; C12N 9/38; C12N 9/76; A61Q 11/00

(52) U.S. Cl. CPC A61K 8/66; C12N 9/22; C12N 9/2471; C12N 9/6427; C12Y 304/21064; C12Y 304/21001; C12Y 304/21004; C12Y 302/01023; A61Q 11/00

(57) ABSTRACT

Disclosed are compositions and formulations comprising enzymes or other biocatalyst that cleave surface-accessible DNA polymers and/or glycoprotein carbohydrate chains at galactose residues in dental calculus, and optionally further include one or more proteolytic enzymes, thereby destroying the structural integrity of the calculus, and allowing it to be readily removed without requiring special treatment by a trained dental professional. Also disclosed are methods for removing dental calculus using the disclosed compositions and formulations.

### CROSS REFERENCE TO RELATED APPLICATIONS

The present application claims benefit under 35 U.S.C. §119 of U.S. Provisional Patent Application No. 62/780,565, entitled, “COMPOSITIONS AND METHODS FOR REMOVING DENTAL CALCULI” filed Dec. 17, 2018, the entirety of which is incorporated herein by reference.

### FIELD

The present disclosure relates to compositions and methods for reducing and removing dental plaque and calculus in human and non-human animals.

### BACKGROUND

Dental tartar, also referred to as dental calculus, is a fossilized/mineralized substance that, if not removed, progressively accumulates and ultimately leads to periodontal diseases such as gingivitis and periodontitis resulting in a chronic inflammatory state that can predispose to diabetes or heart disease in humans and non-human animals, including but not limited to dogs and cats. Presently, methods of removing calculus are performed by trained dental professionals at best once or twice a year, and are essentially limited to scraping of the tooth surface using metal alloy dental picks or an ultrasonic device to fragment and dislodge the calculus from tooth enamel. Calculus is not effectively removed by dentifrice formulations such as toothpastes and mouth rinses used in routine daily oral hygiene practice. Thus, a crucial unmet need exists for a product for regular home use that will reduce and remove dental calculus.

### SUMMARY

Among the various aspects of the present disclosure are a first dental composition comprising or consisting essentially of one or more calculus targeting enzymes or other biocatalyst selected from enzymes or other biocatalysts having at least one of the following catalytic activities: (i) hydrolyzes the phosphate ester bonds in deoxyribonucleic acid (DNA); (ii) hydrolyzes glycoprotein carbohydrate polymers at sugars such as galactose, N-acetyl-glucosamine or other sugars, (iii) proteolytic activity that cleaves the amino acid backbone of proteins; and (iv) any combination thereof. The at least one calculus targeting enzyme can be a DNase having a catalytic activity that hydrolyzes the phosphate ester bonds in deoxyribonucleic acid (DNA). In one aspect, the DNase is present in an amount of about 100,000 Kunitz units/mL. In another aspect, at least one calculus targeting enzyme or other biocatalyst in the composition is a beta-galactosidase having a catalytic activity that hydrolyzes glycoprotein carbohydrate polymers at galactose sugars. The beta-galactosidase can be present, for example, in an amount of about 500 units/mL to about 5000 units/mL, or about 2500 units/mL. In other aspects, the dental composition can comprise any one or more enzymes or other biocatalysts having a catalytic activity that hydrolyzes glycoprotein carbohydrate polymers at any one or more of the following: i) at N-acetyl-glucosamine sugars; (ii) at fucose sugars; (iii) at neuraminic acid (sialic acid) sugars.

In another aspect, the present disclosure provides a dental composition comprising a DNase enzyme or other biocatalyst having a catalytic activity that hydrolyzes the phosphate ester bonds in deoxyribonucleic acid (DNA), a beta-galactosidase enzyme or other biocatalyst having a catalytic activity that hydrolyses glycoprotein carbohydrate polymers at galactose sugars, and an enzyme or other biocatalyst having a catalytic activity that hydrolyses glycoprotein carbohydrate polymers at N-acetyl-glucosamine sugars.

In another aspect, the present disclosure provides a dental composition comprising one or more proteolytic enzymes, such as but not limited to trypsin, proteinase K, and chymotrypsin.

In another aspect, the present disclosure encompasses a first dental composition comprising or consisting essentially of enzymes having at least one of the following catalytic activities: (i) hydrolyzes the phosphate ester bonds in deoxyribonucleic acid (DNA); (ii) hydrolyzes glycoprotein carbohydrate polymers at sugars such as galactose, N-acetyl-glucosamine or other sugars, and (iii) any combination thereof, and excluding a proteolytic enzyme as disclosed herein, and a second dental composition comprising or consisting essentially of one or more proteolytic enzymes as described herein. In methods as disclosed herein, the second dental composition can be used in combination with the first dental composition not comprising the proteolytic enzymes as disclosed herein, according to methods as disclosed herein.

In another aspect, the present disclosure provides a dental composition comprising at least two different calculus targeting enzymes selected from (a) a DNase enzyme or other biocatalyst having a catalytic activity that hydrolyzes the phosphate ester bonds in deoxyribonucleic acid (DNA); (b) a beta-galactosidase enzyme or other biocatalyst having a catalytic activity that hydrolyses glycoprotein carbohydrate polymers at galactose sugars; (c) an enzyme or other biocatalyst having a catalytic activity that hydrolyses glycoprotein carbohydrate polymers at N-acetyl-glucosamine sugars; (d) an enzyme or other biocatalyst having a catalytic activity that hydrolyzes glycoprotein carbohydrate polymers at N-acetyl-galactosamine sugars; (e) an enzyme or other biocatalyst having a catalytic activity that hydrolyzes glycoprotein carbohydrate polymers at fucose sugars; and (f) an enzyme or other biocatalyst having a catalytic activity that hydrolyzes glycoprotein carbohydrate polymers at neuraminic acid (sialic acid) sugars.

Any of the disclosed dental compositions optionally further include one or more proteolytic enzymes or other biocatalyst having a catalytic activity that hydrolyzes polypeptides backbones.

In various aspects, a dental composition according to the disclosure may comprise the enzymes (a) and (b), the enzymes (a) and (c), the enzymes (a) and (d), the enzymes (a) and (e), the enzymes (a) and (f), or the enzyme (a) and any combination of two or more enzymes selected from (b), (c), (d), (e) and (f). A dental composition according to the disclosure may comprise any of the disclosed combinations of calculus targeting enzymes (a), (b), (c), (d), (e) and/or (f), optionally in further combination with any one or more of the proteolytic enzymes as disclosed herein. A non-limiting composition may comprise for example a DNase, a beta-galactosidase and a proteolytic enzyme selected from proteinase K, trypsin and chymotrypsin.

Alternatively, a dental composition comprising any combination of (a), (b), (c), (d), (e) and/or (f), may exclude proteolytic enzymes but be used in combination with a second dental composition comprising one or more proteolytic enzymes.

In another aspect, the present disclosure provides a calculus reducing dental formulation comprising any of the disclosed dental compositions, and an orally acceptable carrier or excipient, such as but not limited to a gelling agent.

In another aspect, the present disclosure provides a method for reducing or removing calculus dentalis in humans and non-human animals, including but not limited to dogs and cats, comprising contacting a tooth surface with an effective amount of at least one calculus targeting enzyme selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, and any combination thereof.

In another aspect, the present disclosure provides a method for reducing or removing calculus dentalis in humans and non-human animals, including but not limited to dogs and cats, comprising contacting a tooth surface with any of the disclosed dental compositions or calculus reducing dental formulations. A composition may comprise for example a DNase, a beta-galactosidase and a proteolytic enzyme selected from proteinase K, trypsin and chymotrypsin.

In any of the methods, contacting the tooth surface may comprise contacting the tooth surface with any of the dental compositions or formulations disclosed herein, waiting for a period of time sufficient for calculus disruption to occur, and then removing the dental composition from the tooth surface. Alternatively, contacting the tooth surface may comprise contacting the tooth surface with a first dental composition or formulation as disclosed herein but excluding any proteolytic enzymes, waiting for a period of time sufficient for calculus disruption to occur, removing the first dental composition or formulation followed by contacting the tooth surface with a second dental composition or formulation comprising one or more proteolytic enzymes as disclosed herein, waiting for a period of time sufficient for further calculus disruption to occur, and removing the second dental composition or formulation.

In another aspect, the present disclosure provides a kit comprising components for reducing or removing dental calculus from a tooth surface in humans and non-human animals, including but not limited to dogs and cats, the kit comprising any one or more of the dental compositions disclosed herein. For example, a kit may comprise: (a) a first composition comprising an effective amount of at least one calculus targeting enzyme selected from an enzyme having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, a proteolytic enzyme, and any combination thereof; (b) an effective amount of an oral care agent; and (c) instructions for applying the composition of (a) to the tooth and for applying the oral care agent of (b) to the tooth. Alternatively, a kit may include a first dental composition or formulation comprising at least one calculus targeting enzyme selected from an enzyme or other biocatalyst having a catalytic activity that hydrolyzes the phosphate ester bonds in deoxyribonucleic acid (DNA), an enzyme having a catalytic activity that hydrolyzes glycoprotein carbohydrate polymers at galactose sugars, but excludes proteolytic enzymes as disclosed herein; and a second dental composition or formulation comprising one or more proteolytic enzymes. In certain aspects, the instructions may instruct the order of applying a first and a second dental composition, and/or the order of applying the oral care agent of (b). For example, instructions may provide that the oral care agent of (b) is applied after the composition of (a) is applied to the tooth, or before the composition of (a) is applied to the tooth.

In another aspect, the present disclosure provides kits comprising an amount of any one or more of the disclosed dental compositions or calculus reducing dental formulations in humans and non-human animals, including but not limited to dogs and cats, together with instructions for applying the composition(s) or formulation(s) the tooth.

Other aspects and features of the disclosure are detailed below.

### DETAILED DESCRIPTION

The present disclosure provides new dental compositions and formulations for reducing or removing dental calculus, methods of their use to reduce or remove dental calculus, and kits comprising the compositions and formulations in humans and non-human animals, including but not limited to dogs and cats.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

The term “calculus targeting enzyme” as used herein refers to: (i) an enzyme that hydrolyze(s) the sugar-phosphate ester linkages of deoxyribonucleic acid (DNA); (ii) an enzyme that hydrolyzes glycoprotein carbohydrate at linkages containing galactose, N-acetyl-glucosamine, fucose, N-acetyl-galactosamine, or sialic acid; and/or (iii) a proteolytic enzyme that hydrolyzes amino acid linkages in protein backbone. Calculus targeting enzymes include but are not limited to DNase and beta-galactosidase, and the proteolytic enzymes such as proteinase K and trypsin and others as disclosed elsewhere herein.

As various changes could be made in the above-described cells and methods without departing from the scope of the invention, it is intended that all matter contained in the above description and in the examples given below, shall be interpreted as illustrative and not in a limiting sense.

(I) Dental Calculus

Calculus has a complex composition and will vary between different individuals, dependent on diet, genetics (subtle compositional variations in saliva) and the makeup of the oral microbiome, but the mechanism of its deposition remains to be determined. In a recent report (Aghanashini, S., et al., J. of Health Sciences and Res. (2016) 7:42-50) six theories of calculus formation were reviewed: the Booster Mechanism; the Epitactic concept; the Inhibition theory; the Transformation theory; the Bacterial Theory; and the Enzymatic theory. All but the Bacterial theory focus on inorganic components. The Bacterial theory hypothesizes that bacteria attaching to the tooth surface is responsible for calculus formation. Studies from the United States in the 1960s and 1970s focused on the composition of calculus hydrolysates and did not venture to identify the macromolecular organic components, if any. Since then, further progress in the U.S. has not been forthcoming. Thus, the field has not consolidated around a single view of calculus development.

Recent studies of dental calculus harvested from ancient teeth have assessed for the presence of DNA, and demonstrated that DNA is an extremely rugged molecule. But, in addition to DNA, calculus contains salivary glycoproteins, mucin being the most abundant member. An additional component of calculus formation is the protein meshwork created by the activity of transglutaminase, which crosslinks glutamine residues on one protein with a lysine residue on an adjacent protein molecule. This process is thought to originate at the tooth gingival interface, where surface proteins on the epithelial surface become crosslinked to soluble salivary proteins creating a meshwork that extends from the epithelium to the enamel surface. Thus, at the tooth/gingival interface, proteins on the epithelial surfaces become cross-linked to other salivary and food proteins by the enzyme transglutaminase, creating an additional foundational material for mineralization.

The model informing the present disclosure begins with chemically-clean tooth enamel in the normal oral environment. Without intending to be bound by theory, it is believed that mucin will bind to positively charged calcium ions in the outer surface of the enamel hydroxyapatite (Ca₁₀(PO₄)₆(OH)₂), and that salivary calcium will further bind to the outer face of the enamel-associated mucins, initiating a laminated arrangement. It is further believed that the less abundant but much larger DNA from the oral environment will associate with available hydroxyapatite calcium and with the calcium bound to the outer face of the immobilized mucins. DNA and mucin can be approximated as cylindrical structures with the charged groups arrayed radially. Therefore, only a fraction of the charged groups will occupy a face of the molecule that is interacting with the underlying matrix (¼-5 in the case of DNA). Thus, the effectiveness of cooperative interactions relies upon the molecular length to supply the strength needed to make the interaction of sufficient duration in a highly hydrated environment. By reducing the length of the DNA polymer or cleaving the carbohydrate chains, terminating in negative charges, from the glycoprotein peptide backbone, the strength of the cooperative interactions is reduced. With progressive reduction of DNA polymer length and increased cleavage of carbohydrate chains from glycoprotein peptide backbones, the cooperativity integral to calculus and plaque structure is lost, allowing the constituents to be washed away.

DNA is recognized as highly resistant to cleavage. Chemical hydrolysis of DNA requires boiling it in acid. The relationship of the length of DNA, relative to a bacterial cell, is on a macro-scale, but the role of DNA and mucin in the formation of plaque and calculus is on the molecular-scale. The number of associative events that mucin and DNA can achieve are enormous, contributing to the strength of the calculus aggregate. Thus, DNA's extensive length and chemical imperviousness makes it a strategic component of calculus. While mucin is not as rugged as DNA, it is sufficiently sturdy to play an important structural role. Both mucin's and DNA's association with immobilized Calcium is enhanced through cooperativity. In this context, cooperativity is like the base pairing of double stranded DNA. While the DNA base pair interactions are established by hydrogen bonding (weak, compared with ionic salt bridges that will dominate in calculus) the precise geometry of the double helix, and the large number of base pairs makes separating the two strands difficult enough to require temperatures approaching boiling. In the case of calculus formation, when one salt bridge comes undone, through competition with water, those remaining upstream and downstream salt bridges will retain the two undone bridge partners in close enough proximity to facilitate their rapid re-association.

A purely chemical approach to the removal of calculus would require a chemical environment that would be chemically extreme, resulting in damage to the oral cavity. However, a biologic/enzymatic approach would be a non-toxic, effective and an efficient approach to disintegrating dental calculus. DNA can be cleaved by the enzyme deoxyribonuclease (DNase) anywhere along its length, at accessible sugar-phosphate bonds, reducing the length of the DNA polymer to short stretches of DNA (oligonucleotides) with the proportional loss of cooperativity. The element of the mucin glycoprotein that forms the associations with calcium are the sulfate and sialic acid groups (both negatively charged) at the ends of the carbohydrate chains. The carbohydrate chains of salivary mucin are diverse and large. They terminate in neutral sugars (56%), sialic acid (26%) and sulfate (19%) and vary in average chain lengths of 13 units, 17 units and 41 units, respectively (Thomsson, K. A., Glycobiology (2002) 12: 1-14). The carbohydrate chains are composed predominantly of galactose, fucose, and N-acetylglucosamine with lesser quantities of N-acetyl galactose. The mucin glycoproteins dimerize end-to-end and then go on to form higher-order structures. The microbes of the oral and gut microbiome have evolved to exploit the mucin carbohydrates as a nutrient source. They secrete N-acetyl-glucosaminidase, beta-galactosidase, N-acetyl-galactosidase, fucosidase, neuraminidase (sialidase) to cleave the carbohydrate chains into smaller sizes for nutrient uptake (Derrien, M., Gut Microbes (2010) 1:254-268). While the most accessible region to cleave the carbohydrate chain is in the middle of the chain at the galactose and N-acetyl-glucosamine residues with the enzymes beta-D-galactosidase and beta-N-acetyl-D-glucosaminidase, respectively, any cleavage that removes the acidic sulfates and sialic acid units from the protein chain will eliminate the cooperativity, important in the calculus and plaque architecture.

All of these enzymes are widely expressed by organisms in nature, where they cleave their polymer substrates into components that are readily utilized for the organism's nutrient requirements. Some of these organisms have evolved to successfully occupy and exploit extreme environments, such as hot springs. The evolution will have involved altering enzyme sequences to maintain their three-dimensional architecture and activities in high temperature environments. This will provide these enzymes with greater thermal stability and longer shelf life.

(II) Dental Compositions and Formulations

Compositions and formulations according to the present disclosure comprise at least one calculus targeting enzyme, and may include a combination of two or more calculus targeting enzymes each with different catalytic activities that hydrolyze different chemical constituents in the structure of calculus. Calculus targeting enzymes include enzymes that hydrolyze any one of: (i) the sugar-phosphate ester linkages of deoxyribonucleic acid (DNA); (ii) glycoprotein carbohydrate at linkages containing galactose; (iii) glycoprotein carbohydrate at linkages containing N-acetyl-glucosamine; (iii) glycoprotein carbohydrate at linkages containing fucose; and (iv) glycoprotein carbohydrate at linkages containing sialic acid. The calculus targeting enzymes, both DNases and carbohydrate chain-cleaving enzymes, act upon the DNA and glycoprotein elements that are readily accessible at the surface of dental plaque and calculus. As the surface-accessible DNA is cleaved by DNase into shorter segments, for example, the shorter segments will have lost their capacity for cooperative associations with the underlying matrix, allowing water to displace the cleaved DNA. Similarly, when surface-accessible glycoprotein carbohydrate chains are cleaved, by beta-galactosidase, for example, the underlying sialic acid/sulfate-calcium association becomes singular, i.e., is no longer connected to the protein backbone, and no longer a component of a cooperative structure and is effectively displaced by the abundant water. As the surface-accessible DNA and glycoproteins are cleaved and displaced, the DNA and glycoproteins that were beneath the now displaced cleaved DNA and glycoproteins are now accessible to the DNase and carbohydrate chain-cleaving enzymes that repeat with progressive cycles of cleavage and displacement, thereby disintegrating the three-dimensional plaque and calculus structure, and thus disintegrating the plaque and calculus.

Thus, in one aspect a dental composition according to the present disclosure comprises at least one calculus targeting enzyme selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, and any combination thereof.

In a composition as disclosed, at least one calculus targeting enzyme or other biocatalyst can be a DNase having a catalytic activity that hydrolyzes the phosphate ester bonds in deoxyribonucleic acid (DNA). In one aspect, the at least one calculus targeting enzyme is a DNase. The amount of DNase can be varied in amount from about 50,000 Kunitz units/mL to about 800,000 Kunitz units/mL. In different examples, DNase is present in an amount of about 100,000 Kunitz units/mL, about 200,000 Kunitz units/mL, about 300,000 Kunitz units/mL, about 400,000 Kunitz units/mL, about 500,000 Kunitz units/mL, about 600,000 Kunitz units/mL, about 700,000 Kunitz units/mL, or about 750,000 Kunitz units/mL. A non-limiting example of DNase is Bovine pancreatic deoxyribonuclease (i.e., “Deoxyribonuclease I”) available from Worthington Biochemical Corp. In another aspect, at least one calculus targeting enzyme in a composition is a beta-galactosidase having a catalytic activity that hydrolyzes glycoprotein carbohydrate polymers at galactose sugars. The amount of beta-galactosidase can vary from about 500 Kunitz units/mL to about 20,000 Kunitz units/mL. In one example, beta-galactosidase is present in an amount of about 8,000 Kunitz units/mL. In other examples, beta-galactosidase is present in an amount of about 1,000 Kunitz units/mL, about 2,000 Kunitz units/mL, about 3,000 Kunitz units/mL, about 4,000 Kunitz units/mL, about 5,000 Kunitz units/mL, about 6,000 Kunitz units/mL, about 7,000 Kunitz units/mL, about 9,000 Kunitz units/mL, or about 10,000 Kunitz units/mL. As detailed further below, a composition may include any one or more proteolytic enzymes. The amount of a proteolytic enzyme, such as but not limited to proteinase K or chymotrypsin, can vary from about 500 Kunitz units/mL to about 10,000 Kunitz units/mL. In different examples, a proteolytic enzyme is present in an amount of about 1,000 Kunitz units/mL, about 2,000 Kunitz units/mL, about 3,000 Kunitz units/mL, about 4,000 Kunitz units/mL, about 5,000 Kunitz units/mL, about 6,000 Kunitz units/mL, about 7,000 Kunitz units/mL, about 8,000 Kunitz units/mL, or about 9,000 Kunitz units/mL.

Units of an enzyme as disclosed herein should be understood according to customary usage in the field for each enzyme. For example, for DNase, 1 unit is defined as the amount of enzyme required to produce an increase in absorbance at 260 nm of 0.001/min/mL at 25° C. of highly polymerized DNA, under conditions of HCI, pH 7.5, 50 mM MgCl₂, 13 mM CaCl₂. For Beta-Galactosidase, 1 unit is the amount of enzyme required to hydrolyze 1.0 micromole of o-nitrophenyl Beta-D-galactoside to o-nitrophenyl and D-galactose per minute at pH 7.3 at 37° C., 410 nm. For Proteinase K, 1 unit is the amount of enzyme required to digest urea-denatured hemoglobin at pH 7.5, 37° C., per minute, to produce absorbance equal to that of 1.0 μmol of L-tyrosine using Folin & Ciocalteu's phenol reagent (6). (See, e.g., www.worthington-biochem.com/PROK/cat.html). Those of skill in the art will appreciate how to define a unit for other proteolytic enzymes. In the examples below, the enzymes amylase and DNase-free RNase are used as controls.

A dental composition according to the present disclosure may comprise any one or more enzymes or other biocatalysts having a catalytic activity that hydrolyzes glycoprotein carbohydrate polymers at any one or more of the following: i) at N-acetyl-glucosamine sugars; (ii) at fucose sugars; (iii) at neuraminic acid (sialic acid) sugars. Any one or more enzymes of (i), (ii) or (iii) can be combined in a composition with a DNase and/or beta-galactosidase as detailed above. Thus, in various aspects, a dental composition according to the disclosure may comprise any combination of two or more enzymes disclosed herein. Non-limiting examples of such enzymes are N-acetyl-glucosaminidase, N-acetyl-galactosidase, fucosidase, and neuraminidase (sialidase), each capable of cleaving the carbohydrate chains. The most accessible region for cleaving the carbohydrate chain is in the middle of the chain at the galactose and N-acetyl-glucosamine residues, using the enzymes beta-D-galactosidase and beta-N-acetyl-D-glucosaminidase.

A dental composition disclosed herein optionally comprises at least one proteolytic enzyme. Non-limiting but exemplary proteolytic enzymes are proteinase K, trypsin and chymotrypsin, but any proteolytic enzyme with catalytic activity having broad specificity for amino acid linkages in protein back-bones can be used. Proteolytic enzymes are listed in Table 1 below:

TABLE 1
Proteolytic enzymes
Proteases

Aminopeptidase M
Bromelain
Carboxypeptidase A
Carboxypeptidase B
Carboxypeptidase Y
Cathepsin C
Chymotrypsin
Collagenase
Dispase
Endoproteinase Arg-C
Endoproteinase Asp-N
Endoproteinase Glu-C (S. aureus V. 8)
Endoproteinase Lys-C
Enterokinase
Factor Xa
Ficin
Kallikrein
Papain
Pepsin
Plasmin
Pronase
Proteinase K
Subtilisin
Thermolysin
Thrombin
Trypsin

Stable enzymes for preparing the dental compositions and formulations can be readily obtained as a purified, lyophilized powder from a commercial enzyme supplier such as Worthington Biochemical Corp. (Lakewood, N.J.). Enzymes may be sourced from animal tissue such as animal (e.g., bovine) pancreas, or produced using recombinant methods. The lyophilized powder is dissolved in an aqueous solvent, which may be water. Sufficient solvent is added to the commercially supplied vial of lyophilized powder, to fill the vial about half way, and the remainder of the vial filled with glycerol to produce a reasonably shelf stable 50/50, water/glycerol solution. The solution can be maintained as such in a refrigerator for at least a few weeks. A plastic or glass pipette or other instrument is used to extract about 50 μl to about 200 μl of each enzyme solution. The enzyme or other biocatalyst solution, or combination of enzyme or other biocatalyst solutions, or a composition or formulation comprising the enzymes is then applied to the teeth using any of a variety of known oral application methods or tools, with particular attention paid to the gingival border. For example, the enzyme solution(s) or a composition or formulation containing one or more enzymes can be applied to a toothbrush, or preferably to a smaller interdental pick with a brush. Alternatively, multiple enzymes can be prepared as described, and then combined in a single solution and then applied to the applicator brush, or each enzyme solution can be applied separately to the brush. Alternatively, a composition or formulation comprising one or more enzymes can be prepared as described, and then applied to the applicator brush. The brush is used to apply the enzyme solution to the tooth surfaces, with particular attention paid to the gingival border where calculus tends to form.

Any one or more calculus targeting enzymes can be prepared as a simple solution as detailed above, or combined with orally acceptable additives, carriers or excipients to prepare a liquid, paste or gel form that helps maintain contact of the enzyme(s) with the tooth surface for a more extended period than a liquid allows. Enzyme solutions as disclosed herein can be combined with or added to a mouthwash or toothpaste composition as known in the art. Non-limiting examples of orally acceptable additives, carriers or excipients are generally as known in the art and include thickeners or gelling agents, binders, stabilizers, preservatives, flavorings, fluoride salts, surfactants, abrasives, tartar control agents, calcium sequestrants, and colorings. Additives such as flavorings and colorings can be generally as known in the art and readily commercially available. Non-limiting examples of thickeners and gelling agents are gellan gum (low acyl or high acyl), glycerol, silica, guar gum, xanthan gum, polyethylene glycols, polyvinyl pyrrolidones and co-polymers thereof, polylactic acids, polyglyocolic acids, long chain fatty acid alcohols, cellulose-based polymers and acrylate polymers. Non-limiting examples of carriers are orally acceptable alcohols such as ethanol, isopropanol and glycerol. Non-limiting examples of tartar control agents are generally as known used in readily commercially available dentifrice products, such as pyrophosphates and their salts, polyphosphates, polyphosphonates and mixtures thereof. Pyrophosphate salts include dialkali and tetra-alkali metal pyrophosphate salts and mixtures thereof. Non-limiting examples of antiseptics and preservatives are quaternary ammonium salts, polymers thereof, chlorhexidine and salts thereof, polyhexamethylene biguanide, octenidine, organic acids, chelating agents for example a calcium chelating agent (e.g., Ethylenediaminetetraacetic acid (EDTA)), essential oils, and parabens. Non-limiting examples of antibiotics are penicillin and tetracyclin. Non-limiting examples of orally acceptable abrasives are silica or other inorganic particles, synthetic polymer particles, or organic particles such as plant-derived particles. Non-limiting examples of orally acceptable surfactants are non-ionic, cationic, anionic and zwitterionic surfactants.

(II) Methods of Removing Dental Calculus

The present disclosure encompasses methods for reducing or removing dental calculus from a tooth surface in humans and non-human animals, including but not limited to dogs and cats. A method for reducing or removing calculus dentalis comprises for example contacting a tooth surface in humans and non-human animals, including but not limited to dogs and cats, with an effective amount of at least one calculus targeting enzyme as disclosed herein. The contacting may be for example by applying a solution (e.g., an aqueous solution), or a dental composition or dental formulation as disclosed herein to the tooth surface, waiting for a period of time and then rinsing the mouth out, typically with water. It will be appreciated that the amount of time can vary depending on a range of factors including the degree and severity of the calculus build-up, the individual being treated, whether the treatment is taking place in a professional office by a dental professional or at home, and other factors. For example, the solution can be applied to the tooth surface for a period of less than about 60 minutes, less than about 30 minutes, less than about 15 minutes, less than about 15 minutes, less than less than about 10 minutes, less than about 5 minutes, less than about 2 minutes, less than about 1 minute, or about 30 seconds.

The contacting may be performed on a repeated and/or regular basis, such as once or twice or more often on a daily basis, once every two days once every three days, once every four days, once every five days, once every six days, once weekly, once biweekly, once every three weeks, or once monthly, or once or twice annually at approximately regular spaced intervals. Further, the contacting may be before, during/in combination with, or after, contacting or treatment with an oral care agent such as a toothpaste or mouth rinse. Following the contacting with an enzyme or other biocatalyst solution or composition, disintegrated or loosened calculus can be further removed from the tooth surface and oral cavity by rinsing with water or another agent, manual scaling or scraping, brushing, and/or swabbing.

(III) Kits

A further aspect of the present disclosure provides kits comprising an amount of any of the disclosed dental compositions or formulations, or any combination thereof, as detailed above. A kit optionally includes one or more brushes and/or dental picks for applying any of the enzyme solutions, compositions or formulations to teeth, and then removing disintegrated calculus from the tooth surfaces. Kit components can be provided in suitable containers along with other kit components such as any commercially available containers or packaging and the like. The kits provided herein generally include instructions for carrying out the methods detailed herein. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

### Example 1: Analysis of Dental Tartar/Calculus Composition and Structure

Calculus, harvested from teeth using metal alloy dental picks is placed in 0.5 mL of water in 1.5 mL microfuge tubes. The calculus is pulverized using a Teflon pestle fitted to the microfuge tube. The contents are centrifuged, the supernatant aspirated and re-suspended in 1 mL water, and 100 microliter aliquots are drawn and placed in one or more microfuge tubes. The contents of each tube are centrifuged, supernatant aspirated and then re-suspended in 300 microliters of water in a tube containing one of the following:

Nothing added (Water control)

1600 units of DNase 1

25 units of beta-galactosidase

25 units of beta-galactosidase and 1600 units of DNase 1

10 units of proteinase K

20 units of amylase (control)

50 units of DNase-free RNase (control)

The contents of each tube are mixed and incubated for 30 minutes at about 37° C. The contents are centrifuged and the aliquots removed and analyzed for the presence and amount of protein, DNA and calcium. In addition, the products of the DNase treatment are assessed by western blot for mucin. Because of the polymer length, DNA is expected to be the dominant component in the formation and stabilization of calculus. DNA provides a more three-dimensional structure to plaque and calculus because its length allows it to establish a torturous path with periodic bridging with calcium-associated glycoproteins. Transglutaminase crosslinks adjacent proteins, further stabilizing the calculus. Thus, a combination of DNase and beta-galactosidase is an effective calculus-degrading treatment, in that the combination attacks the components with the greatest length. Because both DNase and beta-galactosidase are proteins, a protease can be added either before or after the treatment with DNase and beta galactosidase. Furthermore, because mucin is a major constituent of saliva, spitting saliva out during glycoprotein-hydrolyzing enzyme treatment can increase the treatment efficacy. Table 2 shows results expected when samples are treated as detailed above.

TABLE 2
Percentages of perceived maximum product yields

| | Protein | DNA | Calcium |
|---|---|---|---|
| Control | 5 | 5 | 5 |
| DNase | 70 | 70 | 70 |
| β-gal | 65 | 65 | 65 |
| β-gal + DNase | 90 | 90 | 90 |
| Prot-K | 40 | 40 | 40 |
| DNase-free RNase control | 10 | 10 | 10 |
| Amylase control | 7 | 7 | 7 |

### Example 2: Calculus Targeting Composition

A composition was prepared as follows: DNase 1 and β-galactosidase were each obtained as a purified, lyophilized powder from Worthington Biochemical Corp. (Lakewood, N.J.). Each enzyme in powdered form was dissolved in about 2 mL of water in a vial having a volume of about 4 mL, and the remainder of the vial filled with glycerol to produce a reasonably shelf stable 50/50, water/glycerol enzyme solution. The solution was maintained in a refrigerator for about 2-3 weeks. When Subject A was ready to treat for calculus, about 100 μl of the solution was extracted with a plastic pipette, and applied to the brush portion of a commercially available GUM® interdental brush, but could equally well have been applied using a similar tool. Subject A used the brush to apply the enzyme solution once daily to the tooth surfaces, paying particular attention to the gingival border. After allowing a few minutes to elapse, Subject A proceeded to use a regular toothbrush to remove disintegrated calculus from tooth surfaces. Subject A repeated once daily application of the enzyme solution as described above for a period of about 3 weeks and noticed pronounced reduction of calculus and plaque.

Although illustrated as a solution, it should be appreciated that a dental composition could take a variety of other forms, for example, a paste or gel or other suitable vehicle for delivering the enzyme or enzymes to the tooth surface.

### Example 3: Dog Calculus Targeting Composition

The following solutions were prepared as follows: DNase 1, β-galactosidase, and proteinase K were each obtained as a purified, lyophilized powder from Worthington Biochemical Corp. (Lakewood, N.J.). DNase 1 and β-galactosidase powders were dissolved in about 2 mL of water in a vial having a volume of about 4 mL, and the remainder of the vial filled with glycerol to produce a reasonably shelf stable 50/50, water/glycerol enzyme solution. The solution was maintained in a refrigerator for about 2-3 weeks.

Into a round bottom flask equipped with a magnetic stirring bar, was placed 0.5 g of pulverized dog calculus. Into the flask was added 10000 U of DNase. The contents were warmed to 37° C. and stirred for 10 minutes. The resulting mixture became colloidal. Then, 500 U β-galactosidase was added into the flask maintained at 37° C. This mixture was stirred for an additional 10 minutes. The amount of pulverized dog calculus in the flask was visibly reduced and the amount of colloid increased. 100 ug of Proteinase K was added and stirred for an additional 10 minutes. The amount of the colloidal dispersion increased and the amount of pulverized dog calculus further reduced by about 70-75%.

The individual disclosure of each and every publication, patent, and patent application cited herein is hereby incorporated by reference in its entirety. In the event of any conflict in meaning between a term used herein and a term contained in an incorporated reference, the term as used herein shall control.

What is claimed is:

1. A dental composition comprising at least one calculus targeting enzyme or other biocatalyst selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, a proteolytic enzyme, and any combination thereof.

2. The dental composition of claim 1, wherein at least one calculus targeting enzyme is a DNase having a catalytic activity that hydrolyzes the phosphate ester bonds in deoxyribonucleic acid (DNA).

3. The dental composition of claim 2, wherein the DNase is present in an amount of about 100,000 Kunitz units/mL.

4. The dental composition of claim 1, wherein at least one calculus targeting enzyme is a beta-galactosidase having a catalytic activity that hydrolyzes glycoprotein carbohydrate polymers at galactose sugars.

5. The dental composition of claim 4, wherein the beta-galactosidase is present in an amount of about 2500 units/mL.

6. The dental composition of claim 4, comprising an enzyme or other biocatalyst having a catalytic activity that hydrolyzes glycoprotein carbohydrate polymers at N-acetyl-glucosamine sugars.

7. The dental composition of claim 4, comprising an enzyme or other biocatalyst having a catalytic activity that hydrolyzes glycoprotein carbohydrate polymers at N-acetyl-galactosamine sugars.

8. The dental composition of claim 4, comprising an enzyme or other biocatalyst having a catalytic activity that hydrolyzes glycoprotein carbohydrate polymers at fucose sugars.

9. The dental composition of claim 4, comprising an enzyme or other biocatalyst having a catalytic activity that hydrolyzes glycoprotein carbohydrate polymers at neuraminic acid (sialic acid) sugars.

10. The dental composition of claim 1, comprising at least one proteolytic enzyme.

11. The dental composition of claim 10, wherein the proteolytic enzyme is selected from proteinase K, trypsin and chymotrypsin.

12. A dental composition comprising a DNase enzyme or other biocatalyst having a catalytic activity that hydrolyzes the phosphate ester bonds in deoxyribonucleic acid (DNA), a beta-galactosidase enzyme or other biocatalyst having a catalytic activity that hydrolyses glycoprotein carbohydrate polymers at galactose sugars, and an enzyme or other biocatalyst having a catalytic activity that hydrolyses glycoprotein carbohydrate polymers at N-acetyl-glucosamine sugars, and optionally further comprises a proteolytic enzyme.

13. A dental composition comprising at least two different calculus targeting enzymes selected from (a) a DNase enzyme or other biocatalyst having a catalytic activity that hydrolyzes the phosphate ester bonds in deoxyribonucleic acid (DNA); (b) a beta-galactosidase enzyme or other biocatalyst having a catalytic activity that hydrolyses glycoprotein carbohydrate polymers at galactose sugars; (c) an enzyme or other biocatalyst having a catalytic activity that hydrolyses glycoprotein carbohydrate polymers at N-acetyl-glucosamine sugars; (d) an enzyme or other biocatalyst having a catalytic activity that hydrolyzes glycoprotein carbohydrate polymers at N-acetyl-galactosamine sugars; (e) an enzyme or other biocatalyst having a catalytic activity that hydrolyzes glycoprotein carbohydrate polymers at fucose sugars; and (f) an enzyme or other biocatalyst having a catalytic activity that hydrolyzes glycoprotein carbohydrate polymers at neuraminic acid (sialic acid) sugars, and (g) optionally further comprises a proteolytic enzyme.

14. The dental composition of claim 13, comprising (a) and (b).

15. The dental composition of claim 13, comprising (a) and (c).

16. The dental composition of claim 13, comprising (a) and (d).

17. The dental composition of claim 13, comprising (a) and (e).

18. The dental composition of claim 13, comprising (a) and (f).

19. The dental composition of claim 13, comprising (a) and (g).

20. The dental composition of claim 13, comprising (a) and any combination of two or more enzymes or other biocatalyst selected from (b), (c), (d), (e), (f) and (g).

21. A calculus reducing dental formulation comprising the composition of claim 19 and an orally acceptable carrier or excipient.

22. The calculus reducing dental formulation of claim 21, comprising a gelling agent.

23. A method for reducing or removing calculus dentalis comprising contacting a tooth surface with an effective amount of at least one calculus targeting enzyme or other biocatalyst selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, a proteolytic enzyme, and any combination thereof.

24. The method of claim 23, wherein the method uses an effective amount of at least one calculus targeting enzyme or other biocatalyst selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, and a proteolytic enzyme.

25. The method of claim 24, wherein the contacting comprises contacting with a composition comprising a DNase, a beta-galactosidase and a proteolytic enzyme.

26. The method of claim 24 or 25, wherein the proteolytic enzyme is selected from proteinase K, trypsin and chymotrypsin.

27. The method of claim 24 or 25, wherein the proteolytic enzyme is chymotrypsin.

28. A method for reducing or removing calculus dentalis comprising contacting a tooth surface with a composition of any of claims 1-20 or a calculus reducing dental formulation of claim 21 or claim 22.

29. A kit comprising components for reducing or removing dental calculus from a tooth surface, the kit comprising: (a) a composition comprising an effective amount of at least one calculus targeting enzyme or other biocatalyst selected from an enzyme or other biocatalyst having a catalytic activity that hydrolyzes the phosphate ester bonds in deoxyribonucleic acid (DNA), an enzyme or other biocatalyst having a catalytic activity that hydrolyzes glycoprotein carbohydrate polymers at galactose sugars, a proteolytic enzyme, and any combination thereof; (b) an effective amount of an oral care agent; and (c) instructions for applying the composition of (a) to the tooth and for applying the oral care agent of (b) to the tooth.

30. The kit of claim 29, wherein the instructions of (c) instruct applying the oral care agent of (b) after the composition of (a) is applied to the tooth.

31. The kit of claim 29, wherein the instructions of (c) instruct applying the oral care agent of (b) before the composition of (a) is applied to the tooth.
